(19) United States
(12) Patent Application Publication
(10) Pub. No.: US 2015/0316199 A1
(43) Pub. Date: Nov. 5, 2015

(54) System for Attaching and Variably Positioning Accessories and Devices on a Surface

(71) Applicant: Neil A. Moore, Columbus, OH (US)

(72) Inventor: Neil A. Moore, Columbus, OH (US)

(21) Appl. No.: 14/641,257

(22) Filed: Mar. 6, 2015

Related U.S. Application Data
(60) Provisional application No. 61/948,756, filed on Mar. 6, 2014.

(51) Int. Cl. F16M 11/18; A61B 19/00

(52) U.S. Cl. CPC F16M 11/18; A61B 19/26

(57) ABSTRACT

A system for attaching, positioning and repositioning accessories and/or devices to vertical or horizontal surfaces such that the attached accessories or devices can be positioned and repositioned as desired. The system is comprised of a system base which interlocks with accessory mounts, which in turn engage and secure the fittings of accessories or devices. The system allows the accessories or devices retained in the mounts to be fixed along the base at a desired location and released and moved to another position along the base as desired. Further the system allows the accessories to be removed from the mounts and replaced as needed.

### CROSS-REFERENCE TO RELATED APPLICATION NUMBER

61/948,756

### FEDERALLY SPONSORED RESEARCH OR DEVELOPMENT

No

### FIELD AND BACKGROUND OF THE INVENTION

This invention relates to a system for attaching, positioning and repositioning accessories and/or devices to a surface, and in particular to surfaces of medical, industrial and home storage cabinets, carts, work tables, work benches and desks such that little of the available work surface area is impeded and the attached accessories or devices can be positioned and repositioned for convenience as desired.

Available counter space in can be very limited in some work environments and in those environments it is important to minimize the counter space consumed by accessories or devices sometimes required to perform work tasks. In those circumstances it would be beneficial to keep accessories or devices close at hand without them consuming scarce counter space.

### SUMMARY OF THE INVENTION

I invented a system for attaching, positioning and repositioning accessories and/or devices to a surface, and in particular to surfaces of medical, industrial and home storage cabinets, carts, work tables, work benches and desks such that little of the available work surface area is impeded and the attached accessories or devices can be positioned and repositioned for convenience as desired. The system is comprised of a system base that can be attached to the surface of a host structure through a variety of means and in a variety of orientations and that interlocks with system accessory mounts and system accessory mounts that interlock with and can be positioned along the base as desired. The position of the accessory mounts can be fixed along the base at a desired location and released and moved to another position along the base as desired. The system accessory mounts have a socket for an accessory or device fitting that allows an accessory or device to be fixed in the mount and released and moved or removed as desired.

Although others have invented systems for attaching accessories to surfaces, various aspects of my system for attaching and variably positioning accessories or devices on a surface are superior because it:
is more versatile
is easier to use
is less difficult to attach
can be removable
can be used on a variety of surfaces
offers many attachment options
can be easily scaled to a wide range of surface and accessory or device sizes ranging from very small to very large
can be used with either vertical or horizontal orientations of the system base
offers the user a significant amount of flexibility in the positioning of accessories or devices
makes repositioning of accessories or devices by the user easy
preserves available work surface and can keep mounted accessories or devices off or away from the work surface
allows more accessories or devices to be attached to a surface simultaneously with each being able to be positioned and repositioned independently as desired

### DRAWINGS

### Figures

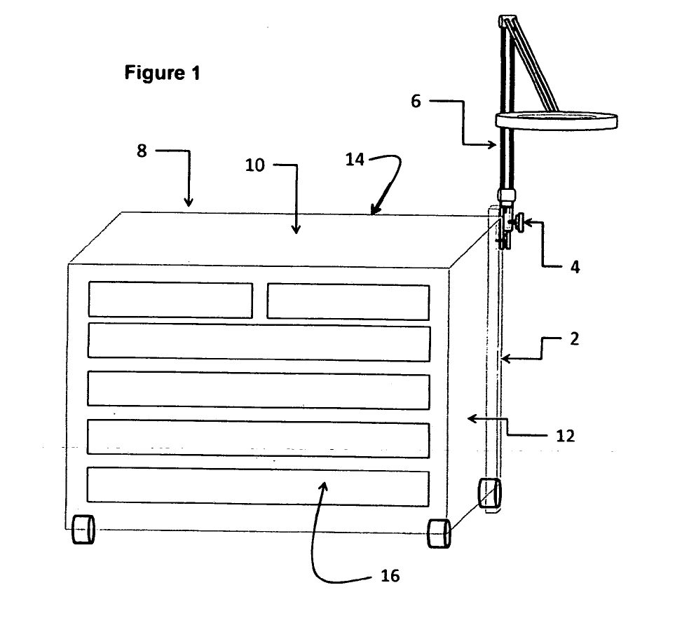

FIG. 1 is a perspective of a wheeled cart with the system for attaching and repositioning accessories or devices on a surface attached to a side wall in a vertical orientation with one system vertical accessory mount and an accessory in place.

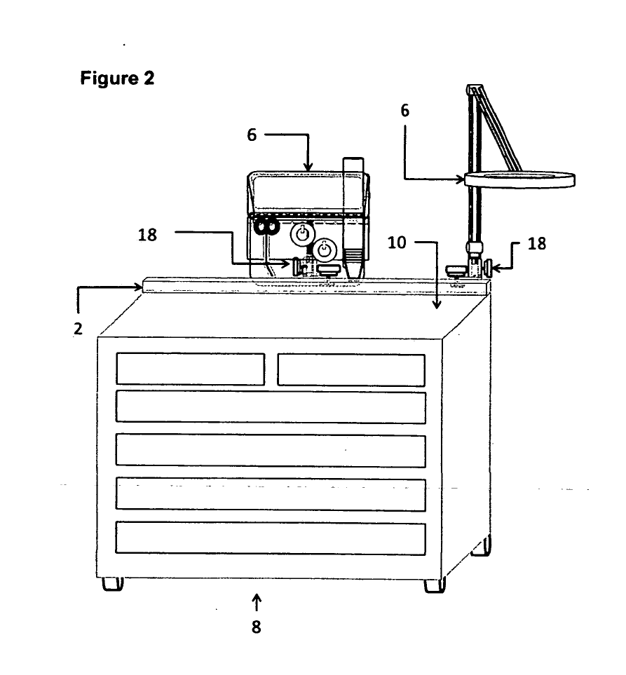

FIG. 2 is a perspective of a wheeled cart with the system attached to the top in a horizontal orientation with two system horizontal accessory mounts and accessories in place.

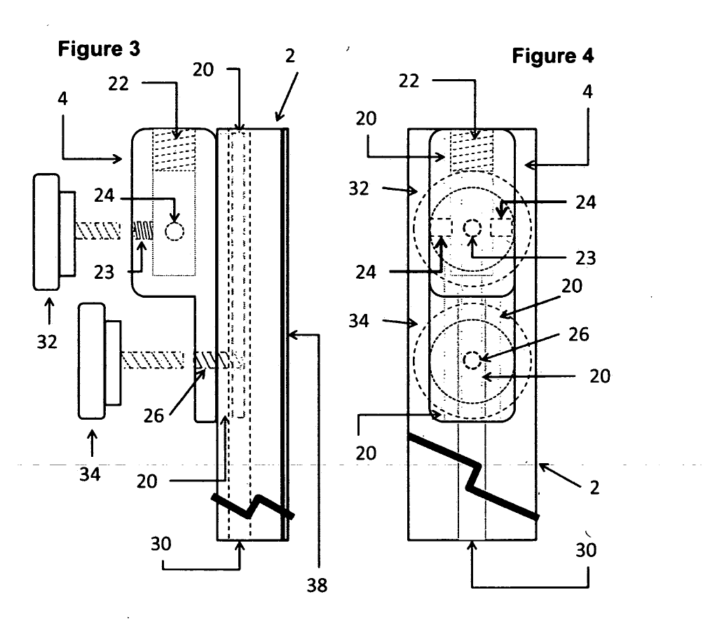

FIG. 3 is a side view of the system with a system vertical accessory mount interlocked with a vertically oriented system base with the accessory mount having manual position-fixing and retaining and accessory retaining methods.

FIG. 4 is a front view of the system with a system vertical accessory mount interlocked with a vertically oriented system base with the accessory mount having manual position-fixing and retaining and accessory retaining methods.

FIG. 5 is a top view of the system with a system vertical accessory mount interlocked with a vertically oriented system base with the accessory mount having manual position-fixing and retaining and accessory retaining methods.

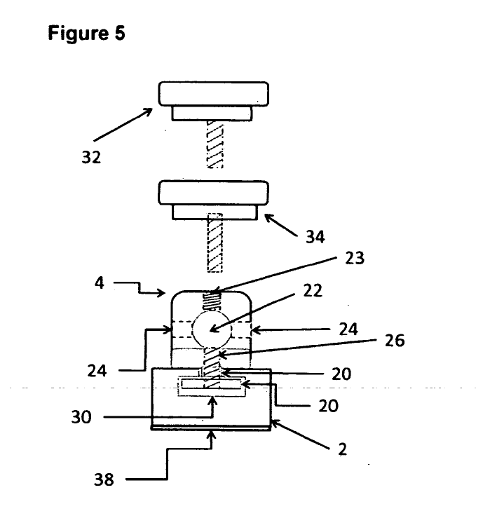

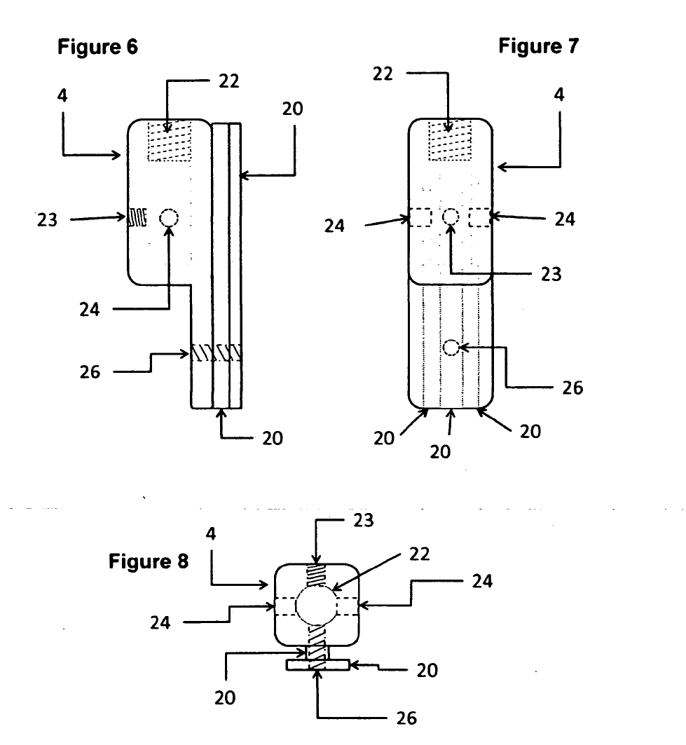

FIG. 6 is a side view of a system vertical accessory mount with a single accessory socket.

FIG. 7 is a front view of a system vertical single socket accessory mount with a single accessory socket.

FIG. 8 is a top view of a system vertical single accessory mount with a single accessory socket.

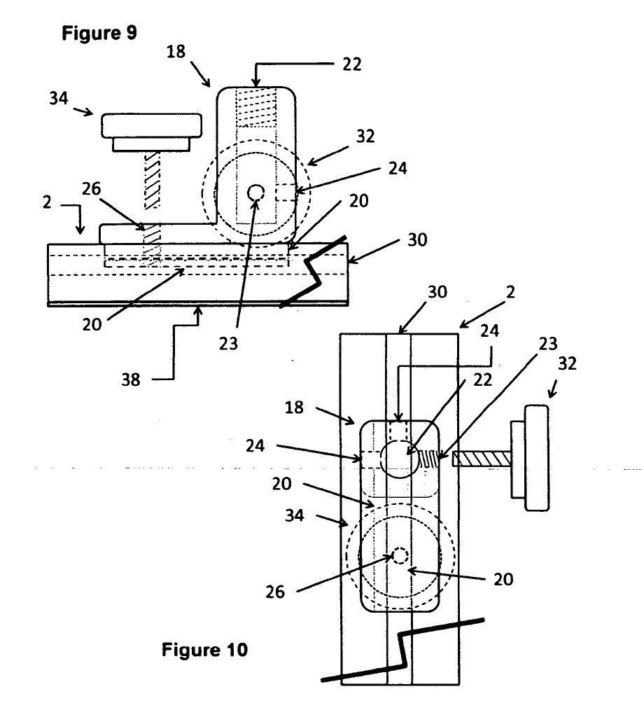

FIG. 9. is a side view of a system with a system horizontal single accessory mount interlocked with a horizontally oriented system base with the accessory mount having manual position-fixing and retaining and accessory retaining methods.

FIG. 10 is a top view of the system with a system horizontal single accessory mount interlocked with a horizontally oriented system base with the accessory mount having manual position-fixing and retaining and accessory retaining methods.

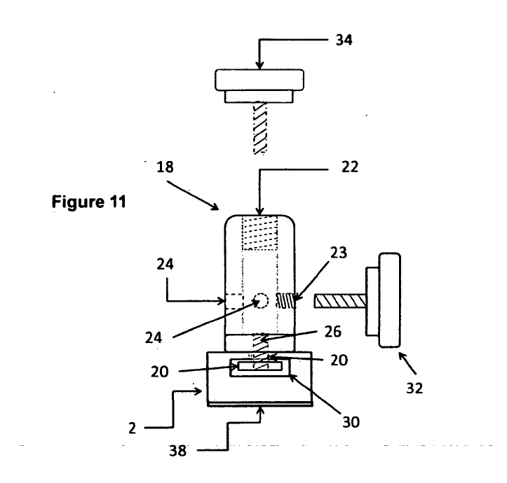

FIG. 11 is an end view of the system with a system horizontal single accessory mount interlocked with a horizontally oriented system base with the accessory mount having manual position-fixing and retaining and accessory retaining methods.

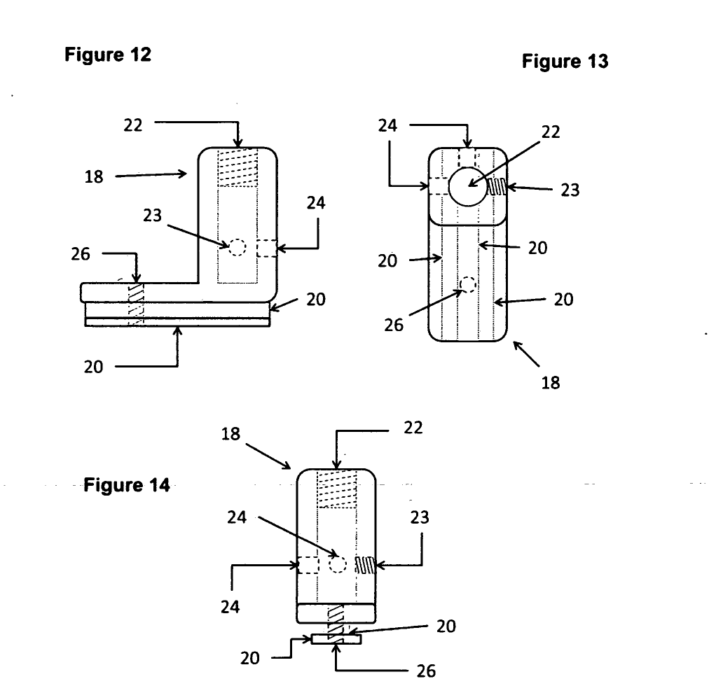

FIG. 12 is a side view of the system horizontal accessory mount with a single socket.

FIG. 13 is a top view of the system horizontal accessory mount with a single accessory socket.

FIG. 14 is an end view of the system horizontal single accessory mount with a single accessory socket.

### DRAWINGS

### Reference Numerals

2. System base with 1 or more accessory mount interlock features
4. System vertical accessory mount
6. Accessory or device mounted in an accessory or device mount
8. Host structure
10. Host structure top
12. Host structure side
14. Host structure back
16. Host structure front
18. System horizontal accessory mount
20. Accessory mount base interlock feature
22. Accessory fitting socket (partially threaded by example)
23. Socket access channel for the accessory fitting retaining method
24. Alternate socket access channel for accessory fitting retaining method
26. Access channel for the base position fixing and retaining method
30. Base accessory mount interlock feature
32. Accessory fitting retaining method
34. Accessory mount base position fixing and retaining method
38. Base attachment method

### DESCRIPTION OF THE DRAWINGS

FIG. 1 is a perspective of a wheeled cart as a host structure 8 with a system base with an accessory mount interlock feature 2 attached to the side of the cart 12 in a vertical orientation. A system vertical accessory mount 4 is interlocked with the base 2, shown in more detail in FIGS. 3,4 and 5, and fixed in its position along the long axis of the base 2. The fitting of an accessory 6 is seated in and retained in the mount 4.

FIG. 2 is a perspective of a wheeled cart as a host structure 8 with a system base with an accessory mount interlock feature 2 attached to the top of the cart 10 in a horizontal orientation. Two system horizontal accessory mounts 18 are interlocked with the base 2, shown in more detail in FIGS. 9, 10 and 11 and fixed in their position along the long axis of the base 2. The fittings of accessories 6 are seated in and retained in the mount 18.

FIG. 3 is a side view of a system vertical accessory mount 4 with a single accessory fitting socket 22 with the accessory mount 4 interlocked with a vertically oriented system base 2 with its associated base attachment method 38, with the accessory mount base interlock feature 20 engaged with the base accessory mount interlock feature 30, the accessory fitting retaining method 32 (removed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed) which passes through the access channel for the base position fixing and retaining method 26 and adjustably engages the structure of the base 2 to reversibly fix the mount 4 in the desired position in the base 2.

FIG. 4 is a front view of a system vertical accessory mount 4 with a single accessory fitting socket 22 with the accessory mount 4 interlocked with a vertically oriented system base 2 with its associated base attachment method 38 (not visible in this view), with the accessory mount base interlock feature 20 engaged with the base accessory mount interlock feature 30, the accessory fitting retaining method 32 (removed and superimposed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed and superimposed) which passes through the access channel for the base position fixing and retaining method 26 and adjustably engages the structure of the base 2 to reversibly fix the mount 4 in the desired position in the base 2. FIG. 5 is a top view of a system vertical accessory mount 4 with a single accessory fitting socket 22 with the accessory mount 4 interlocked with a vertically oriented system base 2 with its associated base attachment method 38, with the accessory mount base interlock feature 20 engaged with the base accessory mount interlock feature 30, the accessory fitting retaining method 32 (removed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed) which passes through the access channel for the base position fixing and retaining method 26 and adjustably engages the structure of the base 2 to reversibly fix the mount 4 in the desired position in the base 2.

FIG. 6 is a side view of a system vertical accessory mount 4 with a single accessory fitting socket 22, an accessory mount base interlock feature 20, a socket access channel 23 (with alternate channel locations identified 24) for the accessory fitting retaining method and the access channel for the base position fixing and retaining method 26.

FIG. 7 is a front view of a system vertical accessory mount 4 with a single accessory fitting socket 22, an accessory mount base interlock feature 20, a socket access channel 23 (with alternate channel locations identified 24) for the accessory fitting retaining method and the access channel for the base position fixing and retaining method 26.

FIG. 8 is a top view of a system vertical accessory mount 4 with a single accessory fitting socket 22, an accessory mount base interlock feature 20, a socket access channel 23 (with alternate channel locations identified 24) for the accessory fitting retaining method and the access channel for the base position fixing and retaining method 26.

FIG. 9 is a side view of a system horizontal accessory mount 18 with a single accessory fitting socket 22 with the accessory mount 18 interlocked with a horizontally oriented system base 2 with its associated base attachment method 38, with the accessory mount base interlock feature 20 engaged with the base accessory mount interlock feature 30, the accessory fitting retaining method 32 (removed and superimposed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed) which passes through the access channel for the base position fixing and retaining method 26 and adjustably engages the structure of the base 2 to reversibly fix the mount 18 in the desired position in the base 2.

FIG. 10 is a top view of a system horizontal accessory mount 18 with a single accessory fitting socket 22 with the accessory mount 18 interlocked with a horizontally oriented system base 2 with its associated base attachment method 38 (not visible in this view), with the accessory mount base interlock feature 20 engaged with the base accessory mount interlock feature 30, the accessory fitting retaining method 32 (removed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed and superimposed) which passes through the access channel for the base position fixing and retaining method 26 and adjustably engages the structure of the base 2 to reversibly fix the mount 18 in the desired position in the base 2.

FIG. 11 is an end view of a system horizontal accessory mount 18 with a single accessory fitting socket 22 with the accessory mount 18 interlocked with a horizontally oriented system base 2 with its associated base attachment method 38, with the accessory mount base interlock feature 20 engaged with the base accessory mount interlock feature 30, the accessory fitting retaining method 32 (removed) which passes through a socket access channel for the accessory fitting retaining method 23 (with alternate channel locations 24 indicated) and adjustably engages the accessory fitting to reversibly retain it in the socket 22, and the accessory mount base position fixing and retaining method 34 (removed) which passes through the access channel for the base position fixing and retaining method 26 and adjustably engages the structure of the base 2 to reversibly fix the mount 18 in the desired position in the base 2.

FIG. 12 is a side view of a system horizontal accessory mount 18 with a single accessory fitting socket 22, an accessory mount base interlock feature 20, a socket access channel 23 (with alternate channel locations identified 24) for the accessory fitting retaining method and the access channel for the base position fixing and retaining method 26.

FIG. 13 is a top view of a system horizontal accessory mount 18 with a single accessory fitting socket 22, an accessory mount base interlock feature 20, a socket access channel 23 (with alternate channel locations identified 24) for the accessory fitting retaining method and the access channel for the base position fixing and retaining method 26.

FIG. 14 is an end view of a system horizontal accessory mount 18 with a single accessory fitting socket 22, an accessory mount base interlock feature 20, a socket access channel 23 (with alternate channel locations identified 24) for the accessory fitting retaining method and the access channel for the base position fixing and retaining method 26.

### DESCRIPTION OF THE INVENTION

In the preferred embodiment, the system base 2 is composed of a suitable aluminum alloy for weight, strength and durability, is formed in the shape of a rectangular parallelepiped or right rectangular prism, is of a length desired or required to meet the need and is attached to a vertical or horizontal surface by means of a suitable adhesive. The attached base 2 has a minimum of 1 accessible system base accessory mount interlock feature 30 that extends the length of its long axis with the feature 30 being a channel of such design that it interlocks with and retains the accessory mount base interlock feature 20 of 1 or more system accessory mounts 4,18 while allowing the mount(s) 4,18 to travel freely along its long axis to a desired position.

The system vertical accessory mount 4 is composed of a suitable aluminum alloy for weight, strength and durability and is interlocked with a vertically oriented system base 2 using the accessory mount base interlock feature 20 such that it is retained by the base interlock feature 30 but can be moved freely along the long axis of the base 2. The accessory mount base interlock feature is a single raised element of the surface of the mount structure that extends along its length and is of such design that it interlocks with and is retained by a correspondingly shaped interlock feature 30 of the base 2. The position of the accessory mount 4 can be fixed in the base 2 by the accessory mount base position fixing and retaining method 34 such that the accessory 6 is conveniently located for the user. The accessory mount base position fixing/retaining method 34 is a manually adjustable knob with an integrated set screw passing reversibly through a threaded access channel 26 in the mount 4 with the access channel 26 located such that it does not intersect with the mount's accessory fitting socket 22. Having been manually advanced through the access channel 26 in the mount 4, the set screw engages the structure of the base accessory mount interlock feature 30 such that it fixes the mount 4 in the desired position along the base 2. The mount's accessory fitting socket 22 is cut or formed into the body of the system accessory mount 4 and is of such as shape and size as to accommodate the fitting of a desired accessory or device. The accessory or device fitting is plugged into the fitting socket 22 and retained in the socket 22 by the accessory fitting retaining method 32. The accessory fitting retaining method 32 is a manually adjustable knob with an integrated set screw passing reversibly through a threaded access channel 23,24 in the mount 4 with the access channel 23,24 located such that passes through the wall of the mount 4 and intersects with the mount's accessory fitting socket 22. Having been manually and reversibly advanced through the access channel 23,24 in the mount 4, the set screw engages the accessory fitting such that it retains the fitting in the socket 22.

The system horizontal accessory mount 18 is composed of a suitable aluminum alloy for weight strength and durability and is interlocked with a horizontally oriented system base 2 using the accessory mount base interlock feature 20 such that it is retained by the base interlock feature 30 but can be moved freely along the long axis of the base 2. The accessory mount base interlock feature is a single raised element of the surface of mount structure that extends along its length and is of such design that it interlocks with and is retained by a correspondingly shaped interlock feature 30 of the base 2. The position of the accessory mount 18 can be fixed in the base 2 by the accessory mount base position fixing and retaining method 34 such that the mounted accessory 6 is conveniently located for the user. The accessory mount base position fixing/retaining method 34 is a manually adjustable knob with an integrated set screw passing reversibly through a threaded access channel 26 in the mount 18 with the access channel 26 located such that it does not intersect with the mount's accessory fitting socket 22. Having been manually advanced through the access channel 26 in the mount 18, the set screw engages the structure of the base accessory mount, interlock feature 30 such that it fixes the mount 18 in the desired position along the base 2. The mount's accessory fitting socket 22 is cut or formed into the body of the system accessory mount 18 and is of such as shape and size as to accommodate the fitting of a desired accessory or device. The accessory or device fitting is plugged into the fitting socket 22 and retained in the socket 22 by the accessory fitting retaining method 32. The accessory fitting retaining method 32 is a manually adjustable knob with an integrated set screw passing reversibly through a threaded access channel 23,24 in the mount 18 with the access channel 23,24 located such that passes through the wall of the mount 18 and intersects with the mount's accessory fitting socket 22. Having been manually and reversibly advanced through the access channel 23,24 in the mount 18, the set screw engages the accessory fitting such that it retains the fitting in the socket 22. Although the invention has been described in connection with a preferred embodiment, it should be understood that various modifications, additions and alterations may be made to the invention by one skilled in the art without departing from the spirit and scope of the invention as defined in the claims.

### Operation:

A system base 2 is attached to a surface using a suitable adhesive 38 in an orientation that meets the needs of the user (ex. horizontally on the top of a medication dispensing cabinet). One or a plurality of accessory mounts 4,18 is/are interlocked 20,30 with the base 2, moved along the base to the desired location(s) and fixed in position by tightening a knob/set screw combination 34 integrated into the mount. The fitting(s) of a desired accessory or device 6 (ex. a lighted magnifier) is put into the accessory fitting socket 22 of the accessory mount 4,18 and a knob/set screw combination 32 integrated with the mount is tightened retaining the accessory or device 6 fitting in the socket 22. If desired, the mount 4,18 and its mounted accessory or device 6 can be repositioned along the base 2 by loosening the set screw 34 that is fixing the mount's position in the base 2, moving the mount 4,18 and mounted accessory or device 6 to a more desirable position along the base 2 and re-tightening the set screw 34. The accessory or device 6 can be moved in the mount 4,18, removed or replaced as needed by loosening the accessory retaining set screw 32 and moving the accessory or device 6, removing its fitting from the socket 22 or and replacing it with the fitting of another accessory or device 6. Additional system mounts 4,18 and accessories or devices 6 may be added to the base 2 as desired.

### ALTERNATE EMBODIMENTS

There many envisioned alternate embodiments of the system base 2. These include but are not limited to alternate shapes including cuboidal, cylindrical and various geometric prisms (ex. pentagonal prism, hexagonal prism, heptagonal prism, octagonal prism and so on), alternate structural shapes including circular and curvilinear, alternate designs including with rounded edges and/or both practical and artistic surface modifications, and alternate compositions including other metal alloys, nylons, plastics and polymers whose physical and performance characteristics would be structurally suitable to retain and support the desired number of accessory mounts and their associated accessories, and bearings or materials with a low coefficient of friction integrated with the base 2 such that they facilitate movement of the accessory or device mounts 4,18 and their envisioned alternate embodiments along the base 2.

There are many envisioned alternate embodiments of the system base attachment method 38. These include but are not limited to clamping brackets that integrate with the base and clamp to the host structure such that the base is attached in a desired orientation, screws and bolts that pass through channels cut through or are formed into the base and magnets which are integrated into the base allowing it to be attached magnetically to a metallic surface of a host structure 8.

There are many envisioned alternate embodiments of the system base interlock feature 30. These include but are not limited to one or more channels in the base of various simple or complex geometric shapes which would be complementary to the shape of the accessory mount base interlock feature 20, one or more fixed or moveable raised elements that interlock with and are retained by complimentarily shaped interlock channels in alternate embodiments of the accessory mount base interlock feature 20 and magnets which are integrated into the structure of the base 2 that retain the accessory mount 4,18 and their envisioned alternate embodiments through cooperative magnetic attachment.

There are many envisioned alternate embodiments of the accessory mount base position fixing and retaining method 34. These include but are not limited to bolts that pass through the mount 4,18 and engage nuts which both interlock with the base accessory mount interlock feature 30 and fix the mount in its position along the base 2 when tightened, cam locks which interlock with the base accessory mount interlock feature 30 and fix the mount in its position along the base when rotated, spring-tensioned elements that interlock with the base interlock feature 30 and can be compressed or lifted to allow the mount 4,18 to be positioned along the base 2 and released to fix the mount 4,18 in position, one or more channels in the mount 4,18 that interlock with a raised interlocking feature of the base 30 and cooperative magnetic attachment.

There are many envisioned alternate embodiments of the system vertical accessory mount 4. These include but are not limited to simple and complex alternate shapes including cuboidal, cylindrical and various geometric prisms (ex. pentagonal prism, hexagonal prism, heptagonal prism, octagonal prism and so on), variations of the angle of its accessory socket(s) 20, methods for modifying the angle of the accessory socket(s) 20 as needed, alternate practical and artistic designs and surface modifications, alternate compositions including other metal alloys nylon, plastics and polymers whose physical and performance characteristics would be structurally suitable to retain and support the desired accessories with or without the inclusion of additional structural components, and alternate configurations including multiple powered and/or unpowered accessory sockets 20 of various embodiments, sockets that include a data connection, and bearings or materials with a low coefficient of friction integrated with the mount body such that they facilitate movement along the base 2.

There are many envisioned alternate embodiments of the shape of the system horizontal accessory mount 18. These include but are not limited to simple and complex alternate shapes that include cuboid, cylindrical and various geometric prisms (ex. pentagonal prism, hexagonal prism, heptagonal prism, octagonal prism and so on), variations of the angle of its accessory socket(s) 20, methods for modifying the angle of the accessory socket(s) 20 as needed, alternate practical and artistic designs and surface modifications, alternate compositions including other metal alloys, nylons, plastics and polymers whose physical and performance characteristics would be structurally suitable to retain and support the desired accessories with or without the inclusion of additional structural components and alternate configurations including multiple powered and unpowered accessory sockets 20 of various embodiments, sockets that include a data connection, and bearings or materials with a low coefficient of friction integrated with the mount body such that they facilitate movement along the base 2.

There are many envisioned alternate embodiments of the accessory mount base interlock feature 20. These include but are not limited to bolts that pass through the mount 4,18 and engage nuts which interlock with the base accessory mount interlock feature 30, cam locks which interlock with the base accessory mount interlock feature 30, spring-tensioned elements that interlock with the base interlock feature 30 and can be compressed or lifted to allow the mount 4,18 to be positioned along the base 2 and released to fix the mount 4,18 in position, one or more channels in the mount 4,18 that interlock with a raised interlocking feature of the base 30 and cooperative magnetic attachment. There are many envisioned alternate embodiments of the accessory fitting socket 22. These include but are not limited to alternate shapes including cuboidal, cylindrical and various geonietric prisms (ex. pentagonal prism, hexagonal prism, heptagonal prism, octagonal prism and so on), threaded and partially threaded cylinders into which a complementarily threaded accessory fitting could be screwed, alternate physical, structural and surface designs as may be required by an accessory or desired for artistic/aesthetic reasons, and separate interchangeable components of the accessory mount 4,18. Other envisioned alternate embodiments include sockets with interchangeable inserts of varying designs designed to allow a common socket shape to accept a variety of accessory or device fitting configurations, electrically powered sockets of various designs to transfer electrical energy to mounted accessories or devices 6, motorized sockets of various designs to transfer kinetic energy to mounted accessories or devices 6, and sockets that include a data connection.

There are many envisioned alternate embodiments of the accessory fitting retaining method 32. These include but are not limited to friction between the socket 22 or an interchangeable socket insert and the accessory fitting, a threaded insert into which a threaded accessory fitting is screwed, a spring and bearing integrated into the wall of the socket 22 or interchangeable socket insert that exerts pressure against the accessory fitting, a magnet or magnets integrated into one or more walls or the base of the socket or interchangeable socket insert.

The embodiments of the invention in which the exclusive property or privilege is claimed are defined as follows:

1. A system for attaching, variably positioning and repositioning accessories or devices to a surface, and in particular to the surface of medical, industrial and home storage cabinets, carts, work tables, work benches and desks such that a minimum of available surface area is rendered unusable and the attached accessories or devices can be positioned and repositioned as desired comprised of:
a system base with a base attachment method through which it is secured to the storage cabinet, cart, work table, work bench or desk with said base having a single or plurality of accessible base accessory mount interlock features which interlock with and retain a single or plurality of system accessory mounts while allowing the interlocked accessory mount(s) to be moved freely along the long axis of the base; and
a system vertical accessory mount that will retain an accessory or device, interlock with a vertically oriented system base, slide freely along the base, can be reversibly fixed in a desired position in the base and repositioned in the base as desired; and
a system horizontal, mount that will retain an accessory or device, interlock with a horizontally oriented system base, slide freely along the base, can be reversibly fixed in a desired position in the base and repositioned as desired.

2. The system base of claim 1, wherein said system base is comprised of:
a structure whose construction and material are of sufficient strength and size to retain the desired accessory mounts and their associated accessories or devices without deformation or structural failure while in use; and
a single or a plurality of accessory or device mount interlock features which can be interlocked with corresponding accessory mount base interlock feature(s) such that the mount is retained by the system base while being positioned and repositioned freely along the base's long axis; and
a single or plurality of smooth or threaded channels passing through the system base through which a base position fixing and retaining method may pass

3. The system vertical accessory mount of claim 1 wherein said vertical accessory mount is comprised of:
a structure whose construction and material are of sufficient strength and size to be retained in the system base while retaining the desired accessory without deformation or structural failure while in use; and
a single or a plurality of base interlock features which can be interlocked with corresponding accessory mount interlock feature(s) of a system base such that the mount is retained by the system base while being positioned and repositioned freely along the base's long axis; and
a single or plurality of threaded, partially threaded or smooth accessory or device fitting sockets aligned in a parallel fashion with the long axis of the mount that will accept and retain an accessory or device fitting; and
a smooth or threaded access channel that is not obstructed by the accessory fitting socket(s) or their fitting retaining methods and which is oriented perpendicularly to the long axis of mount and passes through the mount and its system base interlock feature through which an adjustable base position fixing and retaining method will pass; and
one or more smooth or threaded orifices that extend from the outer surface of the mount at a location adjacent to the wall of each accessory or device fitting socket to the cavity of the adjacent fitting socket through which a accessory fitting retaining method can pass

4. The system horizontal accessory mount of claim 1 wherein said horizontal accessory or device mount consists of:
a structure whose construction and material are of sufficient size and strength to be retained in the system base while retaining the desired accessory or device without deformation or structural failure while in use; and
a single or a plurality of base interlock features which can be interlocked with corresponding accessory mount interlock feature(s) of a system base such that the mount is retained by the system base while being positioned and repositioned freely along the base's long axis; and
a single or plurality of threaded, partially threaded or smooth accessory fitting sockets aligned in a perpendicular fashion to the long axis of the mount that will accept and retain an accessory or device fitting; and
a smooth or threaded access channel that is not obstructed by the accessory or device fitting socket(s) or their fitting retaining methods and which is oriented perpendicularly to the long axis of mount and passes through the mount and its system base interlock feature through which an adjustable base position fixing and retaining method will pass; and
one or more smooth or threaded orifices that extend from the outer surface of the mount at a location adjacent to the wall of each accessory or device fitting socket to the cavity of the adjacent fitting socket through which a accessory fitting retaining method can pass.
